(19) United States
(12) Patent Application Publication
(10) Pub. No.: US 2007/0055102 A1
(43) Pub. Date: Mar. 8, 2007

(54) ENDOSCOPE APPARATUS

(76) Inventor: Yasuo Hirata, Tokyo (JP)

Correspondence Address: OSTROLENK FABER GERB & SOFFEN, 1180 AVENUE OF THE AMERICAS, NEW YORK, NY, 100368403, US

(21) Appl. No.: 11/467,991

(22) Filed: Aug. 29, 2006

(30) Foreign Application Priority Data
Sep. 2, 2005 (JP) JP2005-254915

(51) Int. Cl. A61B 1/00

(52) U.S. Cl. 600/152; 600/130

(57) ABSTRACT

An endoscope apparatus with a simple construction, in which a flexing portion at the tip of an inserting portion has excellent assembly properties and which has reliable bending manipulability, is provided. In the endoscope apparatus, an elongated inserting portion, provided with a flexing portion which is operated with fluid pressure in the vicinity of a tip that includes an observing unit, is inserted inside an object under examination to carry out observation, wherein the flexing portion includes a plurality of tube members whose tips are closed off and whose rear ends are connected to a supply source of pressurized fluid, and an elastic member provided with storage sections in which the respective tube members are stored for controlling the expansion in the circumferential direction over the entire length due to the pressurized fluid.

### BACKGROUND OF THE INVENTION

1. Field of the Invention

The present invention relates to an endoscope apparatus which can be employed in, for example, industrial endoscopes or medical endoscopes. The endoscope apparatus includes a flexing portion, which is operated with an actuator that uses pneumatic pressure or the like, in the vicinity of the tip of an inserting portion to be inserted into an object to be examined.

This application is based on Japanese Patent Application No. 2005-254915, the content of which is incorporated herein by reference.

2. Description of Related Art

Generally, endoscope apparatuses which can be used in industry and medicine, for example, include an elongated inserting portion to be inserted into an object under examination, such as the interior of a lumen. In this type of endoscope apparatus, a flexing portion is disposed close to the tip of the inserting portion. By bending this flexing portion, it is possible to orient an observation surface of a CCD or the like, which is provided at the tip of the inserting portion as an observation device, in any direction.

Therefore, in conventional endoscope apparatuses, a pneumatic actuator that is operated with fluid pressure, such as air pressure, is provided close to the tip of the inserting portion for bending the flexing portion described above. They are configured so that the pneumatic actuator is operated by supplying fluid pressure and discharged air, to enable the observation surface at the tip to be oriented in a desired observation direction.

Such a pneumatic pressure actuators include devices which bend the flexing portion provided, for example, at the tip of a medical catheter in a desired direction. In this case, at the circumferential portion of a flexible tube forming the catheter main body, a plurality of pressure tubes are fixed by welding or adhesive so as to be wound over the entire length in the longitudinal direction. Therefore, tubes formed of pressure-expansion flexible material (for example, silicone, urethane, etc.) which expands and elongates due to the pressurized fluid, such as air, are disposed at the parts of the pressure tubes which are positioned at the flexing portion,

Therefore, by selecting the tube which receives the supplied pressurized fluid to cause it to expand and elongate, the plurality of tubes disposed at the outer circumferential part of the flexible tube function as a pneumatic actuator which bends the flexing portion of the flexible tube in a desired direction. (For example, see Japanese Unexamined Patent Application, Publication No. Hei-6-125868.)

However, the pneumatic actuator disclosed in the above-described patent document uses bonding and the like as the method of fixing a plurality of single tubes to the outside of a catheter main body. Therefore, there are problems with operability and assembly aspects, such as the time required for assembly, and thus, there are demands to improve this situation in a straightforward way.

Moreover, if a bendable tube (single tube) which has pressure expansion characteristics is not reliably fixed to the flexible tube, the behavior becomes unstable when the pneumatic actuator is expanded and elongated; for example, it moves in an unexpected direction. Therefore, it is difficult to reliably bend the flexing portion in the desired direction.

Thus, in convention endoscope apparatuses which include an inserting portion configured to bend a flexing portion with a pneumatic actuator, there are demands to simplify the assembly of the tube functioning as the pneumatic actuator and to also enable a stable bending operation.

### BRIEF SUMMARY OF THE INVENTION

The present invention has been conceived in light of the circumstances described above, and an object thereof is to provide an endoscope apparatus with a simple construction, in which a flexing portion at the tip of an inserting portion has excellent assembly properties and which has reliable bending manipulability.

To solve the problems described above, the present invention employs the following solutions.

In the endoscope apparatus according to the present invention, an elongated inserting portion, provided with a flexing portion which is operated with fluid pressure in the vicinity of a tip that includes an observing unit, is inserted inside an object under examination to carry out observation. The flexing portion includes a plurality of tube members whose tips are closed off and whose rear ends are connected to a supply source of pressurized fluid, and an elastic member provided with storage sections in which the respective tube members are stored for controlling the expansion in the circumferential direction over the entire length due to the pressurized fluid.

With such an endoscope apparatus, the flexing portion includes the plurality of tube members whose tips are closed off and whose rear ends are connected to a supply source of pressurized fluid, and the elastic member which includes storage sections in which the respective tube members are stored for controlling, over the entire length thereof, the expansion in the circumferential direction due to the pressurized fluid. Therefore, even though they are not fixed by bonding or the like, the expansion in the circumferential direction of the tube members to which the pressurized fluid is supplied to expand and elongate them is controlled to stably elongate in the longitudinal direction. Thus, it is possible to bend the flexing portion in a desired direction.

In the endoscope apparatus described above, the storage sections are preferably formed in the shape of indentations whose cross-sectional shapes in the axial direction have openings at an outer circumferential side. By doing so, the tube members are reliably controlled in three directions in the circumferential direction, and the tube members can easily be removed and attached from the outer circumferential side.

In this case, the storage sections preferably include outer-circumferential control portions which close the openings at the outer circumferential side at intervals. By doing so, the expansion at the outer circumferential side in the circumferential direction can be controlled while maintaining the ability to remove and attach the tube members, and it is possible to prevent their motion in the longitudinal direction.

In the endoscope apparatus described above, the elastic member is preferably a plastic molded object. Accordingly, it is possible to easily obtain an elastic member with the desired shape.

In the endoscope apparatus described above, the storage sections are preferably formed such that an outer circumferential surface of each lumen of a multilumen tube is cut away continuously or at intervals. By doing so, it is possible to easily obtain an elastic member having storage sections of the desired shape.

In the endoscope apparatus described above, the elastic member preferably has a slit formed in the axial direction at an outer circumferential side of each lumen of a multilumen tube. By doing so, it is possible to easily form storage sections which can also control the expansion at the outer circumferential side in the circumferential direction while maintaining the ability to remove and attach the tube members.

With the present invention described above, even though they are not fixed by bonding or the like, the expansion in the circumferential direction of the tube members which are supplied with pressurized fluid to expand and elongate them can be controlled in the storage sections and can stably elongate in the longitudinal direction (axial direction), thus allowing the flexing portion to be bent in a desired direction. Therefore, the endoscope apparatus which includes the inserting portion for bending the flexing portion with a pneumatic pressure actuator does not required a process such as bonding or the like in the flexing portion. Thus, assembly of the tube members (tube) functioning as the pneumatic pressure actuator, their replacement during maintenance, and so on are simplified, and in addition, a notable advantage is afforded in that stable flexing can be performed.

### BRIEF DESCRIPTION OF THE SEVERAL VIEWS OF THE DRAWINGS

FIG. 1 is a cut-away perspective view of relevant parts showing an example configuration of a first embodiment of a flexing portion of an endoscope apparatus according to the present invention.

FIG. 2A is an exploded perspective view showing a multilumen and a tube member in FIG. 1.

FIG. 2B is a perspective view wherein the multilumen and the tube member in FIG. 1 are assembled.

FIG. 3 is a sectional view of the flexing portion shown in FIG. 1.

FIG. 4 is a perspective view showing an example of the overall configuration of an endoscope apparatus.

FIG. 5 is an exploded perspective view showing relevant parts in FIG. 4.

FIG. 6 is a perspective view showing the assembly of a multilumen and tube members as an example configuration of a second embodiment of the flexing portion of the endoscope apparatus according to the present invention.

FIG. 7 is a sectional view of the flexing portion using the multilumen and the tube members of the second embodiment shown in FIG. 6.

FIG. 8 is a perspective view showing the assembly of a multilumen and a tube member as an example configuration of a third embodiment of the flexing portion of the endoscope apparatus according to the present invention.

### DETAILED DESCRIPTION OF THE INVENTION

An embodiment of the endoscope apparatus according to the present invention is described below based on the drawings.

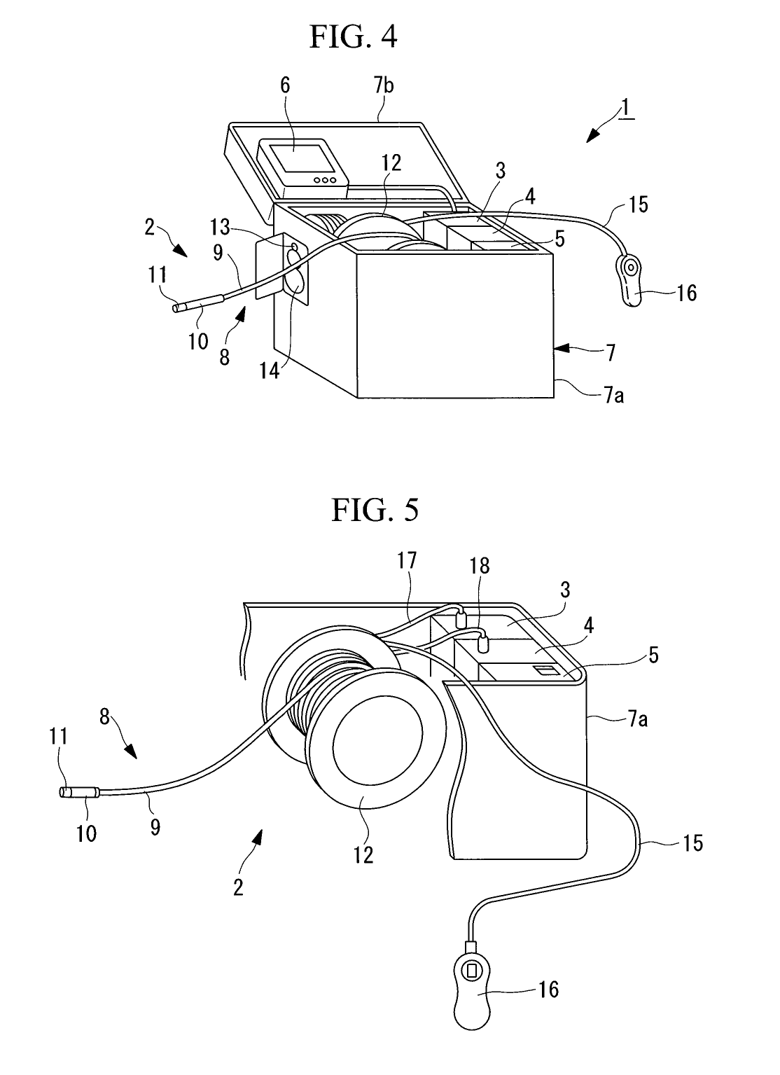

The perspective view shown in FIG. 4 illustrates, in outline, the overall structure of an endoscope apparatus according to this embodiment. This endoscope apparatus 1 is provided with a plurality of constituent elements, including an endoscope main body 2, a CCU (camera control unit) 3, a light-source unit 4, a power supply 5, a monitor 6, and so on. This plurality of constituent elements of the endoscope apparatus 1 are accommodated in a single carrying case (storage case) 7. This carrying case 7 includes a case main body 7a whose top surface is open and a cover member 7b which can open and close the opening at the top surface of the case main body 7a.

As shown in FIG. 5, the endoscope main body 2 includes an elongated inserting portion 8 to be inserted inside an object under examination, for example, a lumen or the like. This inserting portion 8 includes an elongated flexible tube 9 having flexibility, a flexing portion 10 which is coupled near the tip of this flexible tube 9, and a tip-forming portion 11 disposed at the extreme tip. Furthermore, a cylindrical drum 12 onto which the inserting portion 8 of the endoscope main body 2 can be wound up is provided in the carrying case 7. The inserting portion 8 of the endoscope main body 2 is stored inside the carrying case 7 in such a manner that it is wound around the drum 12. Then, the inserting portion 8 of the endoscope main body 2 is drawn out from inside the drum 12 and the carrying case 7 for use, as required.

In FIGS. 4 and 5, reference numeral 13 represents a cylinder accommodating chamber, 14 represents a cylinder, 15 represents a signal line, 16 represents a remote controller, and 17 and 18 represent cables.

### First Embodiment

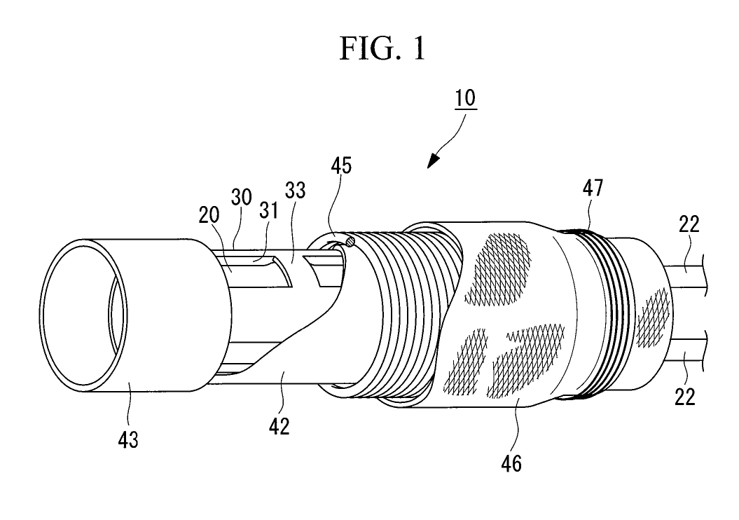

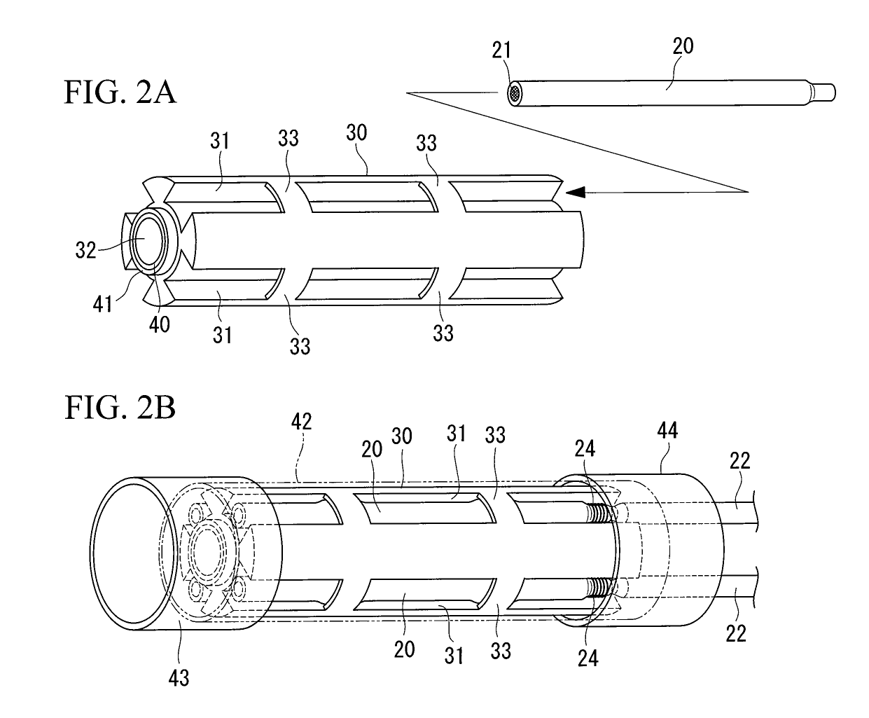

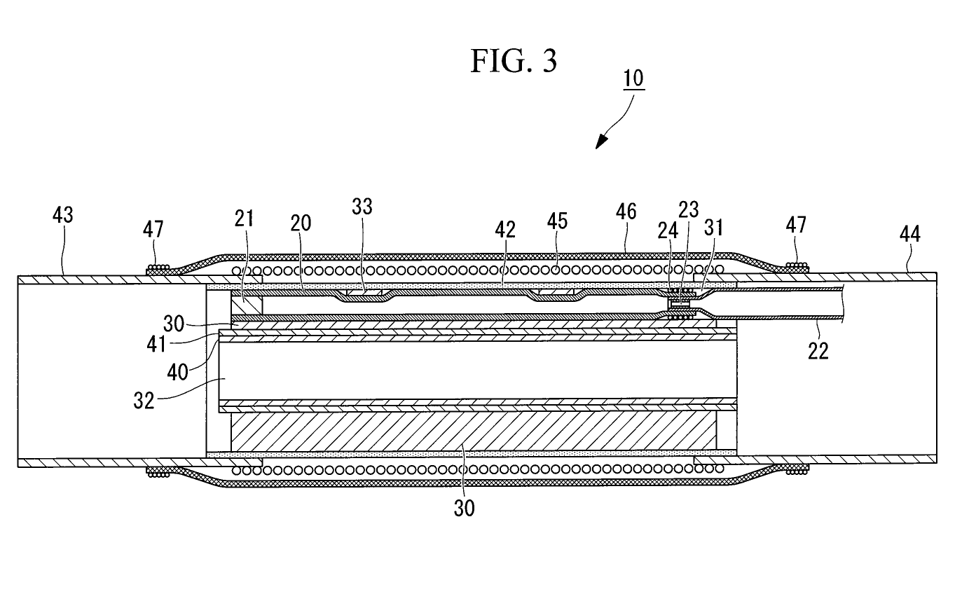

A first embodiment of the configuration of the flexing portion 10 described above is described below with reference to FIGS. 1 to 3.

The flexing portion 10 is disposed close to the tip of the tip-forming portion 11, which is provided with an observation device (for example a CCD) that is not shown in the drawing, and is operated with fluid pressure, such as pneumatic pressure or the like. When the elongated inserting portion 8 is inserted inside the object under examination, the flexing portion 10 orients an observation surface of the tip-forming portion 10 in a desired direction to perform observation. This embodiment is explained using an example in which the fluid pressure for operating the flexing portion 10 uses pneumatic pressure stored inside the cylinder 14.

The flexing portion 10 includes tube members 20 formed of silicone rubber, for example, serving as pneumatic actuators which expand and elongate with pneumatic pressure. In the example shown in the drawing, storage sections 31 are formed on the outer circumferential surface of a multilumen tube 30, which is described later, at four positions with a pitch of 90° in the circumferential direction, and a total of four tube members 20 are disposed, one in each storage section 31. The tube members 20 are closed off at the tips thereof by sealing members 21, and the rear ends thereof are connected to a supply source of pneumatic pressure (pressurized fluid) via respective independent air tubes 22. The sealing members 21 and the tube members 20 are fixed together by, for example, an adhesive or a tie.

The coupled portions of the tube members 20 and the air tubes 22 are provided with pipe members 23 at the innermost side for preventing deformation. After overlapping the air tubes 22 and the tube members 20 in this order so that they cover the outer circumferential surface of these pipe members 23, for example, ties 24 are finally applied to the outer circumferences of the tube members 20 to secure them. Because the pipe members 23 are provided in order to prevent blockage of the supply of pneumatic pressure at the coupled portions where the tie 24 is applied, pipes made of rigid metal or plastic are used.

The multilumen 30 is a molded elastic member formed, for example, of plastic or the like. A through-hole 32 is formed at the central part in the axial direction to serve as a space for wiring or the like for connecting to the observation device and so forth at the tip-forming portion 11, and the storage sections 31 described above are formed at the outer circumference at a 90-degree pitch.

The cross-sectional shape of the storage sections 31 in the axial direction is in the form of a substantially rectangular indentation whose cross-sectional area is optimized according to the cross-sectional shape of the tube members 20 in order to control the expansion of the tube members 20 in the circumferential direction over the entire length. In other words, the storage sections 31 are formed in the shape of an indentation which surrounds the tube members 20 in three directions and controls their expansion in the circumferential direction. In the example shown in the figure, they are in the shape of indentations which have openings at the outer circumferential side of the storage sections 31, and outer-circumferential control portions 33 which close off the opening at intervals are disposed at a plurality of locations at an appropriate pitch.

In the storage sections 31 in this case, through-holes serving as four storage sections around the through-hole 32 are formed at a 90-degree pitch; the through-holes serving as the storage sections may be cut out from the outer circumferential surface, except for portions forming the outer-circumferential control portions 33.

An inner coil 40 and an inner tube 41, in this order from the inner side, are inserted through the inside of the through-hole 32 to pass through in the axial direction. The inner coil 40 and the inner tube 41 are any type of members that are capable of flexibly bending, and wiring or the like, which is not shown in the drawing, passes through the inside of the inner coil 40.

The tube members 20 which extend in the axial direction are disposed to as to be stored inside the storage sections 31 over the entire length. In such a case, a procedure for fixing the storage sections 31 and the tube members 20, for example, by bonding, is not necessary.

The four tube members 20 stored in the multilumen 30 are covered at the outer circumference thereof by an outer tube 42, and a front sleeve 43 and a rear sleeve 44 are fitted to the two ends in the axial direction of the outer tube 42. In order that the outer tube 42 does not hinder the bending operation of the flexing portion 10, a member that is capable of flexibly bending is selected, whereas the front sleeve 43 and the rear sleeve 44 at the two ends of the flexing portion 10 are rigid members. The tip-forming portion 11 and so forth is connected to the front sleeve 43, and the flexible tube 9 is connected to the rear sleeve 44.

An outer coil 45 covers the outer circumferential side of the outer tube 42 so as to overlap the ends of the front sleeve 43 and the rear sleeve 44, and the outer circumference thereof is covered by an outer sheath 46. In order that the outer coil 45 and the outer sheath 46 used here do not hinder the bending operation of the flexing portion 10, any members that are capable of flexibly bending are selected. The two ends of the outer sheath 46 are fixed to the front sleeve 43 and the rear sleeve 44, respectively, by applying a tie 47.

When pneumatic pressure is supplied to the tube member 20 which is selected according to the desired bending direction, the flexing portion 10 of the endoscope apparatus 1 constructed in this way expands because the tip is closed off by the sealing member 21. However, when the tube member 20 expands uniformly in the circumferential direction and grows in diameter, it abuts in three directions against the wall surfaces of the storage section 31 which has higher rigidity than the flexible tube member 21 which is capable of bending. Therefore, because the expansion of the tube member 20 in the circumferential direction is controlled, it elongates in the longitudinal direction which is not controlled. At this time, not only does the storage section 31 control the expansion of the tube member 20 in the circumferential direction, but it also functions as a guide for guiding the elongation of the tube member 20 in a predetermined direction. In addition, the outer-circumferential control portions 33 prevent the expanded tube member 20 from moving in the longitudinal direction.

Therefore, because the tube member 20 receiving the pneumatic pressure is reliably elongated along the storage section 31, even though it is not fixed by bonding or the like, the expansion in the circumferential direction is controlled by the storage sections 31, which enables stable elongation in the longitudinal direction and smooth bending of the flexible flexing portion 10 in a desired direction.

Because it is not necessary to fix the tube members 20 by bonding or the like, construction and maintenance operations are simplified. In particular, in maintenance where it is necessary to replace the tube members 20, when the multilumen 30 becomes exposed, it is possible to easily perform the removing and attaching procedure because, among other reasons, the outer circumferential portion of the storage sections 31 are open and the tube members 20 are not bonded.

In addition, because the tube members 20 are not bonded, the tubes expand uniformly around their entire circumference. Therefore, the durability and reliability are improved compared to a structure in which the bonded portion is fixed and thus a portion in the circumferential direction does not expand.

### Second Embodiment

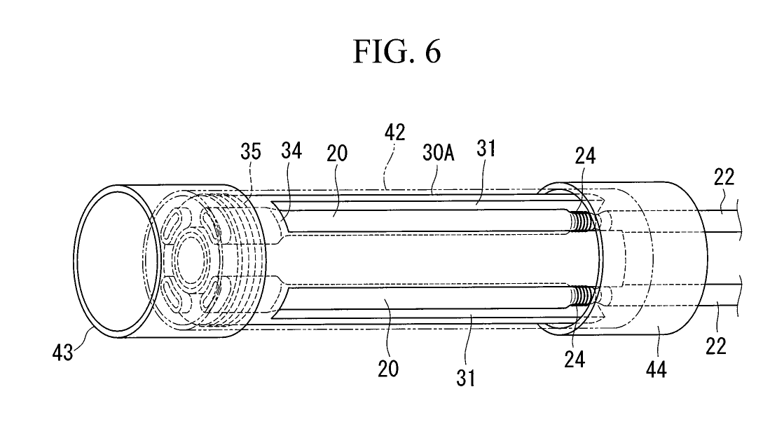

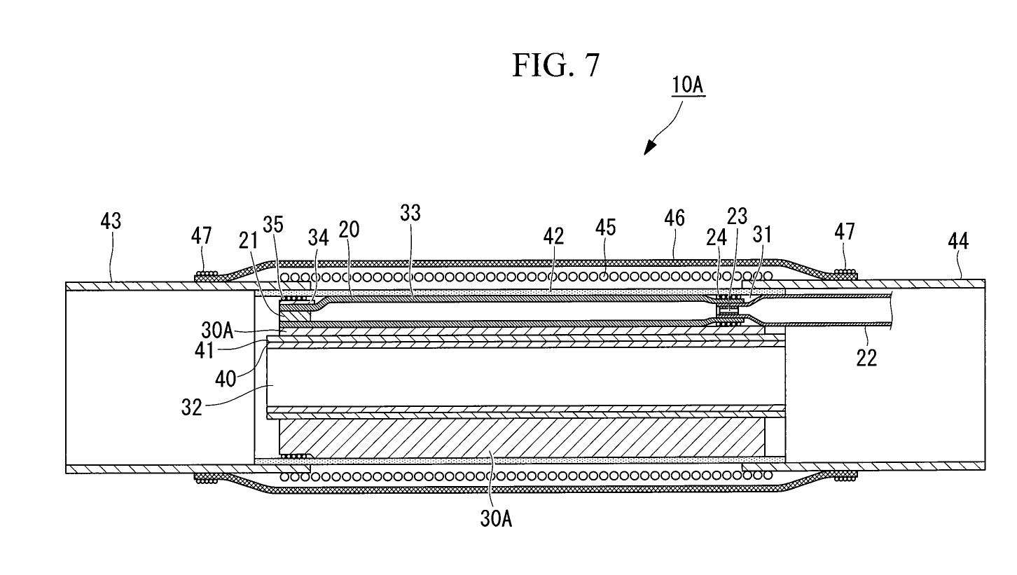

Next, a second embodiment of the configuration of the flexing portion 10 described above will be described based on FIGS. 6 and 7. Parts which are identical to those in the first embodiment described above are assigned the same reference numerals, and a detailed description thereof is omitted.

In this embodiment, the outer circumferential portions of the storage sections 31 formed in a multilumen 30A are open along their entire lengths, except for tip holding portions 34, and members for controlling the opening at the outer circumferential side (the outer-circumferential control portions 33 in the first embodiment) are not provided. In this embodiment, the tube members 20 in the storage sections 31 have their tips inserted inside the tip holding portions 34 of the multilumen 30A and are fixed at the outer circumference by a tie 35. Apart from having their tips fixed inside the tip holding portions 34 by the tie 35, the tube members 20 disposed inside the storage sections 31 are not subjected to any other process for fixing them to the storage sections 31, such as bonding or the like.

When pneumatic pressure is supplied to the tube member 20 which is selected according to the desired bending direction, the flexing portion 10A of the endoscope apparatus 1 having such a configuration expands because the tip side in closed off by the sealing member 21. However, when the tube member 20 uniformly expands in the circumferential direction and grows in diameter, it abuts in three directions against the wall surfaces of the storage section 31, which has a higher rigidity than the tube member 21, and therefore, expansion of the tube member 20 in the circumferential direction is controlled. Accordingly, the tube member 20, whose tip is fixed to the tip holding portion 34 smoothly elongates in the longitudinal direction, which is not controlled. At this time, not only does the storage section 31 control the expansion of the tube member 20 in the circumferential direction, but it also functions as a guide for guiding the elongation of the tube member 20 in a predetermined direction.

Accordingly, because the tube member 20 that receives the supplied pneumatic pressure reliably elongates along the storage section 31, even though it is not fixed by bonding or the like, the expansion in the circumferential direction is controlled by the storage sections 31, which enables stable elongation in the longitudinal direction and smooth bending of the flexible flexing portion 10A in a desired direction.

Because it is not necessary to fix the tube members 20 by bonding or the like, construction and maintenance operations are simplified. In particular, in maintenance where it is necessary to replace the tube members 20, when the multilumen 30A becomes exposed, it is possible to easily perform the removing and attaching procedure because, among other reasons, the outer circumferential portion of the storage sections are open and the tube members 20 are not bonded.

In addition, because the tube members 20 are not bonded, the tubes expand uniformly around their entire circumference. Therefore, the durability and reliability are improved compared to a structure in which the bonded portion is fixed and thus a portion in the circumferential direction does not expand.

### Third Embodiment

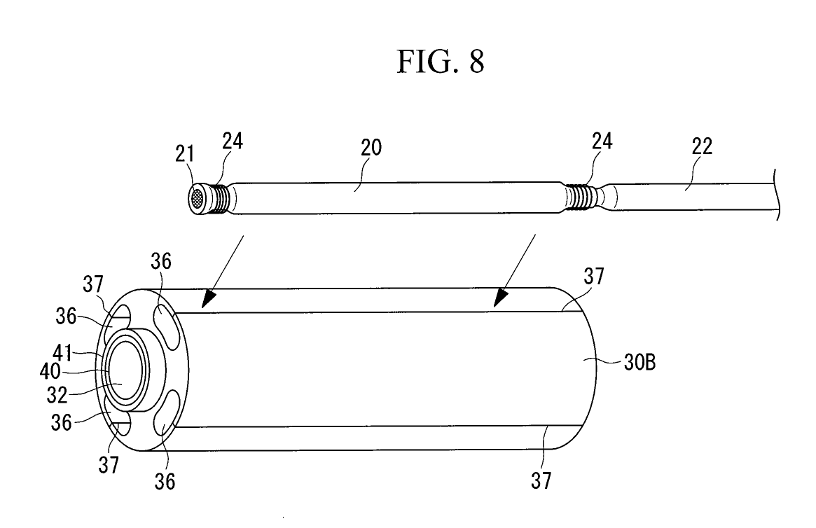

Next, a third embodiment of the configuration of the flexing portion 10 described above will be described based on FIG. 8. Parts that are identical to those in the first and second embodiments described above are assigned the same reference numerals, and a detailed description thereof will be omitted.

In this embodiment, tube passages 36 with substantially elliptical cross-sections, which are formed in a multilumen 30B, have a configuration in which slits 37 reaching the outer circumferential surface are provided. That is, with respect to the tube passages 36 which are disposed at the outer side of the through-hole 32 at equal pitches in the circumferential direction, the multilumen 30B is cut in the longitudinal direction from the tube passages 36 to the outer circumferential surface, thus forming the slits 37. As a result, the tube passages 36, which are storage sections for storing the tube members 20, are effectively sealed around the circumference because the cut surfaces of the slits 37 at the outer circumferential side are sealed off; however, the tube members 20 can still be easily removed and attached by passing them through the slits 37. The tube members 20 do not need to be fixed in the tube passages 36 by, for example, bonding.

When using the multilumen 30B having such a configuration, in the same way as the flexing portions of the first and second embodiments described above, when pneumatic pressure is supplied to the tube member 20 which is selected according to the desired bending direction, it expands because the tip side is sealed off by the sealing member 21. However, when the tube member 20 expands uniformly in the circumferential direction and its diameter grows, it abuts in all directions against the wall surfaces of the tube passage (storage section) 36, which has a higher rigidity than the tube members 21, and therefore, the expansion of the tube member 20 in the circumferential direction is controlled. Accordingly, the tube member 20 elongates smoothly in the longitudinal direction which is not controlled. At this time, not only does the tube passage 36 control the expansion of the tube member 20 in the circumferential direction, but it also functions as a guide for guiding the elongation of the tube member 20 in a predetermined direction. In other words, by molding the multilumen 30B of this embodiment of a flexible plastic or the like and providing the slits 37, it is possible to easily construct storage sections which can also control the expansion in the outer circumferential direction while maintaining the ease with which the tube members 20 are removed and attached.

According to the endoscope apparatus 1 of this invention, as described above, for the tube member 20 to which pressurized fluid, such as pneumatic pressure, is supplied to expand and elongate it, even though no processing is carried out for fixing it, for example, by bonding, the expansion in the circumferential direction is controlled by the storage sections 31, which enables stable elongation in the longitudinal direction and smooth bending of the flexing portions 10 and 10A in a desired direction. Therefore, with the tube members 20 serving as pneumatic actuators, the endoscope apparatus 1 which includes the inserting portion 8 for bending the flexing portions 10 and 10A does not require any steps for bonding in the flexing portions 10 and 10A. Therefore, a replacement operation or the like during assembly or maintenance of the tube members 20 is simplified, and in addition, a stable bending operation is also possible.

In each of the embodiments described above, the fluid pressure for operating the flexing portions 10 and 10A is pneumatic pressure; however, it is not limited thereto and may use other gas pressure, for example, nitrogen gas.

In each of the embodiments described above, four tube members 20 are disposed in the circumferential direction at a 90-degree pitch. However, they can be suitably selected depending on the bending operation and the bending properties; for example, eight at a 45-degree pitch angle can be provided.

The present invention is not restricted to the embodiments described above. Suitable modifications can be made so long as they do not depart from the spirit of the present invention.

What is claimed is:

1. An endoscope apparatus in which an elongated inserting portion, provided with a flexing portion which is operated with fluid pressure in the vicinity of a tip that includes an observing device, is inserted inside an object under examination to carry out observation,
wherein the flexing portion includes a plurality of tube members whose tips are closed off and whose rear ends are connected to a supply source of pressurized fluid, and an elastic member provided with storage sections in which the respective tube members are stored for controlling the expansion in the circumferential direction over the entire length due to the pressurized fluid.

2. An endoscope apparatus according to claim 1, wherein the storage sections are formed in the shape of indentations whose cross-sectional shapes in the axial direction have openings at an outer circumferential side.

3. An endoscope apparatus according to claim 2, wherein the storage sections include outer-circumferential control portions which close the openings at the outer circumferential side at intervals.

4. An endoscope apparatus according to claim 1, wherein the elastic member is a plastic molded object.

5. An endoscope apparatus according to claim 1, wherein the storage sections are formed such that an outer circumferential surface of each lumen of a multilumen tube is cut away continuously or at intervals.

6. An endoscope apparatus according to claim 1, wherein the elastic member has a slit formed in the axial direction at an outer circumferential side of each lumen of a multilumen tube.
